(19) United States
(12) Patent Application Publication
(10) Pub. No.: US 2015/0032114 A1
(43) Pub. Date: Jan. 29, 2015

(54) Cutting Guide for Generating an Outer Contour for a Joint Endoprosthesis

(71) Applicant: Klaus Dmuschewsky, Hamburg (DE)

(72) Inventor: Klaus Dmuschewsky, Hamburg (DE)

(73) Assignee: WALDEMAR LINK GMBH & CO. KG, Hamburg (DE)

(21) Appl. No.: 14/240,173

(22) PCT Filed: Aug. 24, 2012
(86) PCT No.: PCT/EP2012/066517

§ 371 Date: Jun. 16, 2014

Related U.S. Application Data
(60) Provisional application No. 61/527,885, filed on Aug. 26, 2011.

(30) Foreign Application Priority Data
Aug. 25, 2011 (EP) 11178804.8

(51) Int. Cl. A61B 17/17

(52) U.S. Cl. CPC A61B 17/1764
USPC 606/89

(57) ABSTRACT

A cutting guide with a sagittal guide plate is made available for generating an outer contour on a bone in order to apply a joint endoprosthesis, in particular a femoral component of a knee endoprosthesis. The cutting guide according to the invention can be oriented relative to the bone and has at least two sagittal guide slits for guiding a bone-cutting tool, wherein the sagittal guide slits extend through the sagittal guide plate and are arranged at an angle to each other.

### TECHNICAL FIELD

The invention relates to a cutting guide for generating an outer contour on a bone for attaching a joint endoprosthesis, in particular a femoral component of a knee endoprosthesis.

### PRIOR ART

Osteoarthritis, i.e. excess wear of the joints, results in a significant impairment of quality of life for those affected. This condition, which is associated with significant pain, does not, however, only occur in old age, rather increasingly also in the case of younger patients, for example, caused by excess weight and as a result of sporting accidents. Despite a variety of developed methods of treatment, in many cases ultimately only an artificial joint makes it possible to regain pain-free freedom of movement.

However, these artificial joint implants are also affected by wear which leads to the artificial joint having to be replaced with a new one particularly in the case of younger patients. Such replacement may even be necessary several times under certain circumstances. Each replacement, however, requires the presence of sufficient bone stem and it is important when replacing a joint that it largely maintains the existing bone and ligament structures.

What is referred to as surface replacement was developed in order to achieve this objective. Surface replacement only replaces the joint surface and only requires a relatively minor resection of the bone. The ligament system which encloses the joint can furthermore largely be maintained in the case of this technique. Such surface replacement can be carried out either on one side or on two sides. Here, replacement can be carried out on the respective side of the joint across the entire joint surfaces, e.g. in the case of the hip joint, or also only in sections, e.g. on the lateral or medial femoral condyles and tibia head in the case of the knee joint.

If the joint or surface replacement is carried out in the case of what is referred to as hemiarthroplasty only on one side, once implantation has been carried out, the artificial joint surface has the joint cartilage as a mating surface. In the case of replacement mainly carried out on two sides (total replacement), both opposite joint surfaces are replaced with artificial joint surfaces of joint prostheses which are usually composed of metal, plastic and/or ceramic. Such surface replacement is particularly frequently used in the knee joint. This is carried out both in a uni- and bicondylar manner. However, surface replacement can also by all means be used in the case of other joints such as, for example, the hip joint, the shoulder joint or the ankle joint.

One requirement for optimal functionality of joint replacement is its good positioning and alignment in order to avoid defective positions occurring after the operation such as, for example, knock knees or bowlegs or also correct positioning of the joint endoprosthesis which is different to the real joint. Of course, it is equally important to correspondingly correctly position and align the implant in order to ensure perfect kinematics of the joint. This is above all a challenge for the surgeon if, for example, in the case of a knee joint, only the medial or lateral side of the joint is replaced by a joint endoprosthesis. Perfect positioning and alignment is equally important if an implant, at least on one side, comprises several interacting joint surfaces which are, however, implanted separately from one another. In the case of the knee joint, these multi-part implants have in particular the advantage that they protect on one hand the bone stem and secondly the ligament system of the knee. In contrast to this, a coupled joint replacement generally results in the loss of the anterior and posterior cruciate ligament in the knee.

Cutting guides can be used in order to ensure as exact and rapid positioning as possible when implanting or attaching a joint replacement. These guides are positioned and aligned during operation in each case at the end of the bone where the joint endoprosthesis is supposed to be implanted. The resection of the bone subsequently takes place, for example, with the help of sawing, grating or milling, the cutting direction of which is defined by the guides. The implant is finally anchored on the bony outer contour generated by the resection by virtue of the fact that it is pressed in in a cemented or cement-free manner. After initial press fitting, the bone finally grows into the implant over time.

As a result of the relatively complex geometry of the outer contours to be machined and generated, during generation of the outer contour, a multiple changeover is frequently necessary or it is often necessary to use several cutting guides. However, this has above all two disadvantages. On one hand, the precision which can be achieved in terms of the outer contour to be generated is impaired, among other things, because the outer contours generated after changeover of the guide are not flush with those generated prior to changeover. Secondly, the changeover and/or change of the cutting guides and the renewed positioning and alignment require additional time during the operation. This leads in turn on one hand to an increased risk for the patient, above all as a result of the need for a longer period of anaesthesia and on the other hand to a higher risk of infection as a result of the longer period of exposure of the wound.

### EXPLANATION OF THE INVENTION

The object of the present invention was thus to provide a cutting guide which can be positioned and aligned in as simple a manner as possible so that a changeover is avoided where possible. At the same time, the cutting guide should where possible enable surgery which does not damage tissue.

This object is achieved with a cutting guide and a method as indicated in the independent claims. The dependent claims furthermore define preferred embodiments and further developments of the invention.

In a first embodiment of the present invention, a cutting guide is provided for generating an outer contour on a bone, it being possible to attach a joint endoprosthesis (70), in particular a femoral component of a knee endoprosthesis, to the outer contour. The cutting guide has a sagittal guide plate which can be aligned with respect to the bone, and at least two sagittal guide slits for guiding a bone-cutting tool, the sagittal guide slits passing through the sagittal guide plate and being arranged at an angle to one another. In the case of a knee joint, the sagittal guide plate can be aligned on the bone in such a manner that it is located substantially in the sagittal plane of the body or parallel thereto. In this case, the sagittal guide slits break through the sagittal plane of the body.

The guide slits are designed in accordance with the bone-cutting tool. Saws, drills, graters and/or milling cutters are preferably used as bone-cutting tools in the case of use of the cutting guide according to the invention. The guide slits then have dimensions which enable play-free introduction and movement of the respective bone-cutting tool. They extend through the material of the vertical plates, preferably at least the longitudinal sides of their cross-section being parallel to one another. Even if the cross-section of the guide slits is preferably constant, it is also possible that the slit narrows or widens in its direction of passage. In other words, in the case of a guide slit formed in such a manner, its opening on the tool entry side is then larger than on the tool exit side or vice versa.

The sagittal guide plate has, at least on the side facing the joint or the bone, a substantially sagittally extending lateral surface. In other words, the sagittally extending lateral surface is located on the exit side of the guide slits, i.e. in the direction in which the guide slits of the sagittal guide plate extend through the sagittal guide plate. On this lateral surface, the sagittal guide slits are arranged at an angle to one another. It should be noted here that, in the case of more than two sagittal guide slits, the angles enclosed by the longitudinal directions of adjacent guide slits do not have to be identical, but rather different angles can also be enclosed depending on the outer contour to be generated. Here, the outer contour is in turn defined by the inner contour of the joint endoprosthesis, preferably of the joint surface replacement.

The present cutting guide is particularly suitable for generating substantially facet-like contours. The guide slits are correspondingly preferably arranged in such a manner that the outer contour generated by them at the end of the bone is composed of surfaces which are arranged in a facet-like manner and adjoin one another. If one considers a Cartesian coordinate system to designate the locations of adjacent facets or guide slits, in one plane of which system, i.e. e.g. the Y-Z plane, the first facet or the first guide slit lies, the next one is rotated relative to the first about at least one axis, e.g. about the Z- and/or about the Y-axis.

It is advantageous to provide more than two guide slits in the sagittal guide plate, preferably between four and ten and particularly preferably between six and eight guide slits. The reason for this is that a higher number of guide slits increases the number of facets and enables the generation of a more constant outer contour which better approximates the anatomical joint surface. In short, the cut line thus generated by the guide slits under the joint surface substantially follows its outer contour. The cut line is polygonal as a result of the structural arrangement of the guide slits. An ideally arcuate cut line with a plurality of tangentially aligned cuts is thus approximated. The more polygonal the polygon or the more guide slits present, the better the cut line arranged under the joint surface thus follows the outer contour of the joint surface. The advantage emerges from this that the loss of bone substance is minimised when generating the outer contour. The maximum number of guide slits is produced from the stability, which must be ensured despite the slits, of the guide and easy of handling by the surgeon.

The guide slits which go into the sagittal guide plate are inclined at an angle of 40-80°, preferably 50-70° and particularly preferably 55-65° about an axis which is situated substantially parallel to the slit width. Even if the axis of inclination preferably extends parallel to the slit width, it can also be inclined slightly in relation to the longitudinal direction of the slit opening in order to generate facets which are rotated at two angles to one another. The inclination, as seen from the outside to the joint side of the sagittal plate, preferably extends in the direction of the inside of the arc. The slits, which are aligned in a fan shape in relation to one another on the sagittal plate, are at an angle of 10-30°, preferably 12-28°, particularly preferably 15-25° to one another. In other words, the guide slits with their opening longitudinal direction extend from the arcuate cut line to be generated tangentially radially away from the arcuate cut line without the radial lines having a common point of intersection. The angles between the individual guide slits on average become smaller the more facets are used for approximation to the arcuate ideal line parallel to the anatomical joint surface. Nevertheless, the individual angles can by all means deviate from one another.

As a result of the arrangement described above of the guide slits in the sagittal guide plate, the desired outer contour is generated on the joint to be replaced without a changeover of the cutting guide or the use of additional cutting guides. This has on one hand the advantage that the operating time is reduced and on the other hand the precision of the outer contour is reproducibly increased.

In a further preferred embodiment, the sagittal guide slits are arranged within the sagittal guide plate in such a manner that they substantially do not mutually intersect. Each guide slit thus forms a self-contained opening. A self-contained opening within the meaning of the present invention is present if the opening at least on its tool entry side or its tool exit side does not intersect with the opening of another guide slit. In order therefore to guide the bone-cutting tool from one guide slit to the next, the tool must be completely removed from a guide slit in to order to be able to be subsequently introduced back into another guide slit. In order to generate the continuous facet-shaped outer contour despite the non-intersecting guide slits, the slit must be designed to be correspondingly long so that the bone-cutting tool can separate the bone sections covered by the sagittal guide plate, for example, by inclined introduction.

Guide slits which are spatially separate from one another have the advantage that the bone-cutting tool cannot jam, as could otherwise be the case as a result of the transition between two guide slits. If the tool does in fact jam, its movement acts directly on the cutting guide so that correct positioning and alignment of the cutting guide can no longer be ensured.

In the case of a further embodiment of the present invention, the guide slits are arranged distributed substantially on an arcuate line. Here, the guide slits are preferably not aligned along the arcuate line, rather preferably such that they with their opening longitudinal direction are at an angle to the arcuate line and fan out radially towards the outside relative to the arcuate line. In short, the guide slits are rotated in relation to one another and arranged offset to one another so that an arcuate line emerges in the case of a connection of the centres of area of an intersection. The intersection is produced from two intersecting surfaces. The first surface is formed by the guide slit opening, while the second surface is formed by the cut generated by means of the respective guide slit, more precisely, the cross-section of the cut which generates a part of the facet-shaped outer contour. The amount of intersection of these two intersecting surfaces finally produces the above-mentioned intersection.

As a result of such an arrangement of the guide slits, the freedom of movement of the bone-cutting tool when generating the continuous outer contour is increased. This is enabled by virtue of the fact that the guide slits as seen from the arcuate line can be embodied to the outside up the edge there of the sagittal guide plate. This has the advantage that, in the case of non-intersecting guide slits, the bone tissue present between the guide slits can preferably be separated by inclined introduction of the bone-cutting tool.

In a further particularly preferred embodiment, the cutting guide furthermore has a transversal cleft plate which protrudes from the sagittal guide plate to the side in which the guide slits of the sagittal guide plate extend through the sagittal guide plate. In other words, the transversal cleft plate is located on the side of the sagittal guide plate on which the tool exits from the guide slits of the sagittal guide plate. The transversal cleft plate thus forms together with the sagittal guide plate a type of angular stop. In the case of a knee joint, the transversal cleft plate, after the alignment of the sagittal guide plate on the bone, lies substantially in the transversal plane of the body or parallel thereto.

A transversal cleft plate embodied in such a manner enables simple application and positioning of the cutting guide on the bone on which the outer contour is supposed to be generated. The transversal cleft plate can be embodied here so that it can be introduced into the joint space of the joint before one of the joints is resected. However, it equally lies within the meaning of the invention to introduce the transversal cleft plate firstly into the joint space after the opposite joint surface has already been machined for accommodating a joint endoprosthesis. In such a case, the thickness of the cleft plate can be selected to be greater.

In a further embodiment, there is located in the transversal cleft plate explained above at least one further guide slit which extends through the transversal cleft plate so that it supplements the guide slits of the sagittal guide plate, i.e. reinforces the row of guide slits. Here, the guide slit is preferably arranged in the transversal cleft plate so that it supplements the row of sagittal guide slits preferably towards the outside so that a bone-cutting tool guided through the transversal guide slit generates at least one of the outer-lying end surfaces of the outer contour.

The arrangement of a guide slit in the transversal cleft plate has above all the advantage that the cutting guide has a more compact design as a result of this embodiment. With this embodiment, the cutting guide is supplemented by a guide space without increasing the dimensions of the cutting guide. The more compact potential design of the cutting guide contributes, for attachment of the joint endoprosthesis, to as small as possible access to the joint being opened up and the operation technique being less invasive.

In a further preferred embodiment, the cutting guide also has an anterior guide body through which a guide slit which is substantially parallel to the sagittal guide plate extends and/or at least one guide hole. In the case of a knee joint, the anterior guide body is located, after alignment of the sagittal guide plate on the bone, anteriorly in relation to the tibia mating surface.

A guide slit arranged in such a manner and/or a guide hole arranged in such a manner enable that not only the generation of the outer geometry required for implantation is enabled for attachment of the joint replacement, rather additionally the geometry of the outer contour can be adjusted. For example, joint endoprostheses can have on their inner contour at least one pin and/or one web for which a corresponding recess, preferably a bore or a groove, is to be provided in the desired outer contour of the bone.

By integrating the anterior guide body into the cutting guide, further demands on the outer geometry of the contact surface between bone and prosthesis can thus be satisfied. Due to the fact that all guide slits and guide holes of the cutting guide are arranged positioned in a fixed manner relative to one another and the cutting guide only has to be applied and aligned once, the cutting guide can also advantageously ensure a correct alignment of the individual portions of the outer geometry in the case of the above-mentioned complex geometrical requirements.

In one embodiment of the present invention, the cutting guide has at least one alignment projection which is preferably arranged on the transversal cleft plate, and the vertex thereof serves as a pivot point for alignment of the cutting guide. For this purpose, the surfaces which lead to the vertex are inclined in at least one spatial direction. The alignment projection can thus preferably be brought to rest on the opposite joint side and indeed either on the intact joint or on the joint portion already prepared with a recess for accommodating a joint prosthesis. The alignment projection can be embodied as a protruding edge or punctiform elevation. The cutting guide resting by means of the alignment projection can thus be aligned relative to the bone to be resected by rotation and/or tipping, i.e. where applicable by rotational movements about more than one axis, by means of the alignment projection relative to the bone end.

An alignment projection aligned in such a manner enables simple and precise intraoperative alignment of the implant. In other words, such an alignment projection may be sufficient for the alignment of the implant. Of course, supplementary or other alignment devices can, however, also be used.

In one particularly preferred embodiment of the invention, the guide has at least two contact points, preferably at least one of the contact points projecting. Via these contact points, it is possible to place the cutting guide on the joint surface to be resected. Moreover, one of the contact points is preferably formed as a projecting longitudinal body which projects from the sagittal guide plate, the transversal cleft plate and/or the inside of the anterior guide body. The inside of the anterior guide body is located on the side on which the tool exits side of the sagittal guide plate is also located.

The distance between the guide slits and the contact points defines the depth of the cut line, i.e. how deep under the joint surface the outer contour is generated. Here, this distance is primarily dependent on the material thickness of the joint endoprosthesis to be implanted.

Contact points embodied in such a manner enable a targeted and rapid putting in place of the cutting guide on the joint surface to be resected. At the same time, the positioning or at least the prepositioning of the cutting guide relative to the joint surface is already carried out by this putting in place. A further advantage is produced in that the contact points can serve the purpose of ensuring whether the correct implant side has been chosen as a replacement for the joint or the joint portion. In other words, a check can thus be carried out that a correct implant size has been chosen when, during placing of the cutting guide on the joint surface, the contact points rest against the joint at the points intended for this purpose. If this is not the case, despite operation planning, a different implant size can also be selected at short notice via the putting in place of the associated cutting guide. The contact points thus not only contribute to an optimum alignment of the cutting guide, rather also to a correct selection of the joint replacement to be attached.

In a further embodiment of the present invention, the contact points explained above project out of the sagittal guide plate, the transversal cleft plate and/or the anterior guide body. A marking is present here as a further positioning aid for the cutting guide preferably at at least one contact point.

In particular a projecting contact point, such as, for example, a longitudinal body which extends out of the cutting guide, e.g. a pin provided with a marking, has on one hand the advantages of the contact points of the previously described embodiment, but the additional marking furthermore enables positioning of the outer contour to be generated in the direction of the sagittal guide slits. This is particularly important if the joint endoprosthesis has guide pins or guide fins which engage in the outer contour to be generated. For this purpose, recesses must generally be generated so that the cutting guide must be positioned in advance relative to the outer contour to be generated.

In a further embodiment, the cutting guide has at least one through-hole, preferably three through-holes, for fastening of the cutting guide, the through-hole preferably being arranged in the sagittal guide plate on the inside of the arcuate line. The cutting guide is fastened on the bone of the joint surface to be resected via these through-holes. The through-holes arranged on the inside of the arcuate line ensure here that the cutting guide remains securely fastened relative to the bone even after the separation of joint and bone parts for generating the outer contour. The fastening of the cutting guide via the at least one through-hole is preferably performed via Kirschner wires, clamps, screws and/or nails.

The invention furthermore comprises a method for generating an outer contour on a bone for attachment of a joint endoprosthesis, in particular a femoral component of a knee endoprosthesis. The cutting guide described above is used here to generate the outer contour. The method has the following steps:
Putting in place and positioning the cutting guide on the lateral and/or medial side of the joint to be resected, alignment of the cutting guide relative to the joint surface,
Generating the outer contour with a bone-cutting tool and
Removing the cutting guide.

By putting in place the cutting guide on the lateral and/or medial side of the bone, the correct selection of the joint endoprosthesis can initially be checked by checking a correct positioning of the cutting guide relative to the joint surface. If the selection is not correct, the cutting guide can be removed directly again and replaced by another one. Otherwise, the resection of the joint surface can be continued. If the appropriate cutting guide is put in place and properly positioned, the resection of the bone surface can begin. It should be regarded as particularly advantageous that, as a result of the arrangement of the tool guides on the cutting guide, the resection for generating the desired outer contour is carried out without a relocation or changeover of the cutting guide.

In a further particularly preferred embodiment of the method, the cutting guide is put in place via the at least two contact points on the joint surface to be resected. Here, preferably at least one of the contact points is arranged on a transversal cleft plate which has been pushed-in previously between the two sides of the joints, preferably between at least one femoral condyle and the tibial plateau.

After opening up access to the joint, the joint preferably being supported in a flexion or extension position, the contact points located on the cutting guide facilitate the optimum putting in place of the joint endoprosthesis on the joint surface to be resected. The contact points furthermore facilitate a check as to whether the correct prosthesis size has been selected. This can be recognised on the basis of whether the contact points lie on the joint surface where possible without a gap. Preferably particularly easy to identify anatomical points of orientation are chosen as contact points. If, according to a particularly preferred embodiment of the cutting guide, a transversal cleft plate is furthermore located between the two sides of the joint, checking of the prosthesis size can be additionally improved by contact points which are located spatially further apart.

If in particular three contact points are present on the cutting guide, a correct selection of the implant size can in general be detected immediately since, in such a case, preferably all contact points come to lie on the joint surface. Here, at least one of the contact points preferably lies on an anatomically easy to identify and inspect position and thus serves as a type of reference contact point.

In one preferred embodiment of the method for resection of a knee joint, the contact points of the cutting guide lie on at least one of the following regions of the joint surface to be resected: the contact region where the opposing joint surfaces, preferably the femoral condyle and the tibial plateau, come into contact at approximately 85° flexion; the contact region where the opposing joint surfaces, preferably the femoral condyle and the tibial plateau, come into contact at approximately 0° extension; and/or the region in which one end of the joint endoprosthesis to be implanted, preferably the anterior end, rests against the bone.

In particular the anatomical points of orientation described here can be easily identified intraoperatively. If a position which is adjoined later by one of the ends of the joint endoprosthesis is selected as one of the contact points, it can furthermore be ensured that the prosthesis covers the entire sliding region and thus the entire range of movement of the joint.

In one preferred embodiment of the method, prior to putting in place of the cutting guide, a recess, preferably a step-shaped recess, is introduced into the opposite side of the joint.

The introduction of a preferably step-shaped recess in the opposite side of the joint additionally facilitates the putting in place of the cutting guide on the joint surface to be resected or especially the transversal cleft plate between the two sides of the joint.

In a further preferred embodiment of the method, the cutting device is aligned prior to generation of the outer contour via at least one alignment projection which serves as a pivot point for the cutting guide.

The at least one alignment projection present on the cutting guide is supported after the putting in place and positioning of cutting guide in the joint on the outer contour of the opposite side of the joint. A correct alignment of the joint endoprosthesis can thus be ensured in a simple manner. The alignment projections furthermore enable the correction of an incorrect varus/valgus position and a coordination of the interaction in the case of functionally associated but separate joint running surfaces (e.g. knee joint, hand joint or elbow joint).

A combination of the at least one alignment projection and the recess of the above embodiment improves the alignment in the at least one spatial direction, in particular however also an alignment in two spatial directions which is possible in the case of two alignment projections.

In one particularly preferred embodiment of the method, the cutting guide, after putting in place, positioning and alignment via through-holes, is fastened on the bone of the joint to be resected.

The fastening of the cutting guide after putting in place, positioning and alignment ensures that during the generation of the outer contour on the bone the cutting guide maintains its position relative to the bone. This naturally also applies to the generation of a potentially present guide hole or an additional groove in the outer geometry which has been generated or is still to be generated by the resection of the joint surface. The fastening of the cutting guide is carried out here, for example, with Kirschner wires, clamps, screws and/or nails which enable particularly rapid and efficient fastening of the cutting guide.

In a further preferred embodiment of the method, a bone-cutting tool is introduced in each case into the guide slits in order to resect the joint and simultaneously, via the cuts carried out in this case, generate the desired outer contour for attachment of the joint endoprosthesis.

A particular advantage as a result of the use of the cutting guide according to the invention is produced in that the resection of the joint surface and the generation of the desired outer contour are carried out with one and the same cut. In addition to the only one-off putting in place of the cutting guide, this also reduces the operating time required for the resection of the joint surface.

In a further particularly preferred embodiment of the method, prior to and/or after the generation of the outer contour for insertion of the prosthesis via the at least one anterior guide hole, i.e. via the guide hole arranged in the anterior guide body, a hole for accommodating a projection located on the endoprosthesis and/or via the anterior guide slit, i.e. via the guide slit arranged in the anterior guide body, a groove for accommodating a fin located on the endoprosthesis is introduced into the bone.

The use of supplementary cutting guides is enabled in particular as a result of this embodiment of the method since, in addition to the outer contour, recesses already described above can additionally be generated in the outer geometry. These enable the accommodation of potentially present pins or fins on the inner contour of the endoprosthesis which is in contact with the outer contour after implantation.

### BRIEF DESCRIPTION OF THE FIGURES

The invention is explained below on the basis of exemplary embodiments represented in the enclosed figures. In these figures:

FIG. 1 shows a perspective view of the anterior side of the cutting guide,

FIG. 2 shows a further perspective view of the anterior side of the cutting guide,

FIG. 3 shows a side view of the anterior side of a cutting guide according to the invention,

FIG. 4 shows a top view of a cutting guide according to the invention with a pivot point for alignment of the implant in one spatial direction,

FIG. 5 shows the putting in place of a cutting guide according to the invention on one side of the joint,

FIG. 6 shows the putting in place of the cutting guide in a top view and an anterior side view on a knee joint,

FIG. 7 shows a side view of an implanted joint endoprosthesis, i.e. in this case of a joint surface replacement, using the example of a knee, and

FIG. 8 shows a side view of a cutting guide according to the invention from the perspective of the joint to be resected.

FIG. 9 is a schematic view for the arrangement of the guide slits in the sagittal guide plate.

### DETAILED DESCRIPTION OF PREFERRED EMBODIMENTS

Preferred embodiments of the present invention are explained below on the basis of the associated figures. Unless stated otherwise, the same reference numbers generally designate the same features or features with the same effect of the present invention.

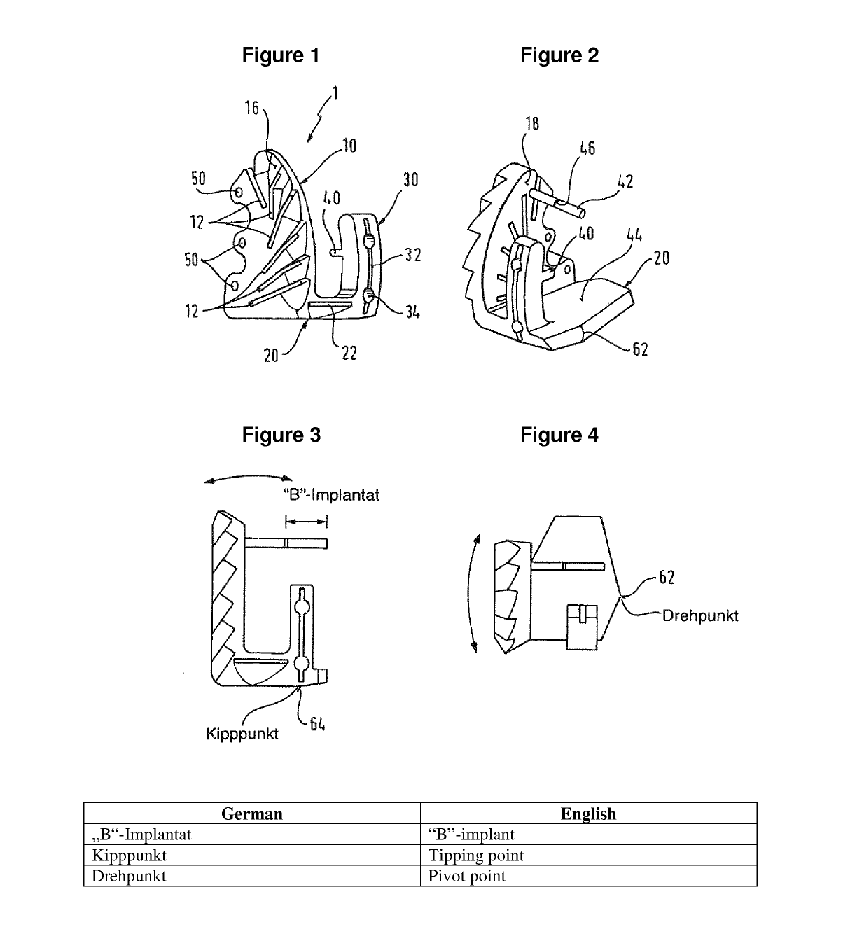

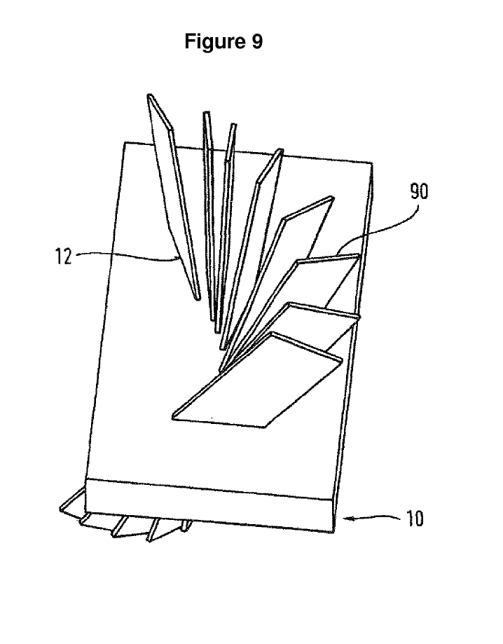

FIG. 1 shows an embodiment according to the invention of cutting guide 1. Both sagittal guide plate 10 and the anterior end of transversal cleft plate 20 and anterior guide body 30 are represented here. The sagittal guide plate has, as shown in FIGS. 2 and 8, a sagittally extending lateral surface 18. Guide slits 12 exiting from lateral surface 18 are arranged in rows on an arcuate line 14 represented in FIG. 8. Sagittal guide slits 12 are arranged both rotated in relation to one another and offset to one another and indeed such that they fan out to the outside from the perspective of the arcuate line. Guide slits 12, 22 and 32 shown in FIG. 1 are formed as guide slits for a saw, in particular an oscillating saw. This is shown by way of example in FIG. 9. Reference number 90 designates a schematically indicated oscillating saw blade which is introduced into guide slits 12. In order to illustrate the arrangement of the individual guide slits inclined at an angle to one another, a saw blade 90 is located in each guide slit 12. Each of these saw blades is inclined about an axis running substantially parallel to the slit width. The guide slits extend on the sagittal guide plate tangentially in relation to an imaginary arcuate line and protrude at an angle from one another. As described above, it is nevertheless equally possible to embody the guide slits for a bone-cutting tool such as a milling cutter.

The guide slits can be embodied as self-contained slits as represented in FIG. 1 in the case of lower sagittal guide slits 12. It is, however, equally possible to embody it open towards the outside of the sagittal plate, as shown, for example, in the case of the sagittal guide slit in the upper region of FIGS. 1 and 2. Moreover, the guide slits in no way have to have the same length, rather can by all means be embodied with different lengths.

FIGS. 1 and 2 furthermore show that sagittal guide slits 12 are indeed arranged at an angle to one another, but are not always rotated by the same angle to one another. Moreover, introduction surfaces 16 for the bone-cutting tool, which facilitate the introduction of the tool into the guide slits and furthermore preferably increase the available guide surface on one side, are provided in each case at the outer ends of sagittal guide slits 12. In one preferred embodiment of the invention, the ends of introduction surfaces 16 serve as an orientation aid when introducing the tool into the guide slits. The ends of introduction surfaces 16 can thus preferably be configured such that the longitudinal axis of the tool, e.g. of the oscillating saw, during introduction into a guide slit is preferably at a right angle to the end edge of introduction surface 16. This also ensures a short operating time and a reproducibly high quality when generating the outer contour on a bone.

Three through-holes 50, via which cutting guide 1 is fastened on joint 80 to be resected (see FIG. 6), are arranged on the inside of arcuate line 14 in FIG. 1. Through-holes 50 are preferably all arranged in a region which, during fastening of cutting guide 1, does not fall in the bone region of the resection. Even if fastening holes 50 in the exemplary embodiment shown extend in the same spatial direction as sagittal guide slits 12, it is equally possible to align it in a different spatial direction or also provide it on a different portion of cutting guide 1.

As represented in FIGS. 1 to 6, a transversal cleft plate 20 is arranged perpendicular to sagittal guide plate 10. Of course, it is also possible to arrange transversal cleft plate 20 at a different angle. The decisive factor is above all the alignment and positioning of the guide elements to one another, i.e. of guide slits 12, 22, 32 and guide holes 34. As shown in FIG. 1, anterior guide slit 22 is arranged in relation to sagittal guide slits 12 so that it supplements the row of sagittal guide slits 12 in the lower region with a further guide slit in terms of technical effect. In the exemplary embodiment shown, guide slit 22 forms the tool guide for generating a facet surface, which lies at the outer end, of the outer contour.

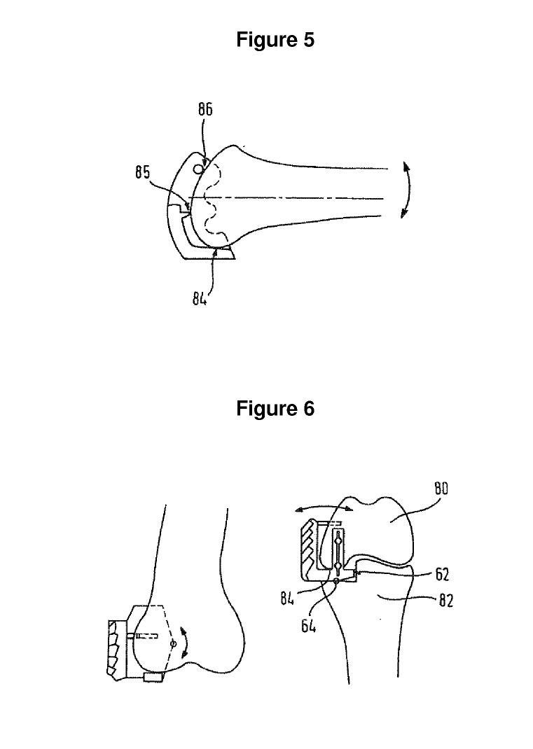

Transversal cleft plate 20 is preferably embodied as shown in FIGS. 1, 2, 4, 5 and 6 so that it projects beyond sagittal guide plate 10 and anterior guide body 30. It is thus ensured that it can be introduced as far as possible into the space between the opposing sides of the joint 80, 82. Transversal cleft plate 20 furthermore has a contact point 44 which, by way of example in FIG. 5, adjoins orientation point 84 which is anatomically easy to identify as a result of its position.

FIGS. 2 and 4 show a pivot point 62 which in this exemplary embodiment according to the invention is embodied as a projecting edge. There is located on the side opposite contact point 44 of transversal cleft plate 20 a tipping point 64, which in the exemplary embodiment shown in FIG. 3 is not formed as an edge, but rather as a punctiform projection which projects out of transversal cleft plate 20.

The mode of operation of the alignment projections, i.e. both of pivot point 62 and of tipping point 64, becomes clear on the basis of FIG. 6. The alignment projection of pivot point 62 and/or of tipping point 64 can be formed as a projecting edge or as a projecting point bearing. During alignment of cutting guide 1, tipping point 64, also enables, for example, where necessary, a correction of an incorrect varus-valgus position by corresponding tipping of cutting guide 1 prior to generation of the outer contour of the bone. In addition to the bearing on the joint cartilage and as shown in FIG. 6, it is also possible to embody the alignment via tipping point 64 when cutting guide 1 lies on an already operatively generated recess in the opposite side of the joint. The same applies to pivot point 62 which in the embodiment shown in FIG. 6 additionally allows an alignment of cutting guide 1 about a second rotational axis. When using cutting guide 1 for resection of a femoral condyle, this enables an alignment of the cutting guide and thus of the joint endoprosthesis to be attached in the rotational direction about the longitudinal axis of the femur.

Moreover, anterior guide body 30 is formed as a further portion of cutting guide 1 according to the invention in FIGS. 1 to 6. Anterior guide body 30 embodied here has one contact point 40, two guide holes 34 and one guide slit 32. It should be noted here that anterior guide body 30 generally has at least one of these features, but it is no way absolutely essential that all these features are present. Both guide slit 32 and guide holes 34 are aligned perpendicular to one another in relation to sagittal guide slits 12. It is, however, by all means possible to select an angle which deviates from this, preferably between 60 and 120°, for example, if such an angle is required for an adjustment of the outer contour of the bone to the inner contour of the joint endoprosthesis. A contact point 40 embodied as a projection is located on the tool exit side of guide holes 34 or of guide slit 32. Contact point 40 is embodied here as an adjoining edge. It can, however, equally be embodied as a surface, such as is the case, for example, in the case of contact point 44 of transversal cleft plate 20 or also as a bearing point (not shown).

Contact point 44 embodied as a surface of transversal cleft plate 20 lies in relation to the joint surface of joint 80 to be resected at point of orientation 84 which is anatomically easy to identify. In a similar manner, contact point 40 of anterior guide body 30 adjoins anatomical point of orientation 85. In the example shown, anatomical point of orientation 84 is the region of contact between the joint surfaces of the knee joint with 85° flexion and point of orientation 85 is the region of contact between femur and tibial plateau with 0° extension.

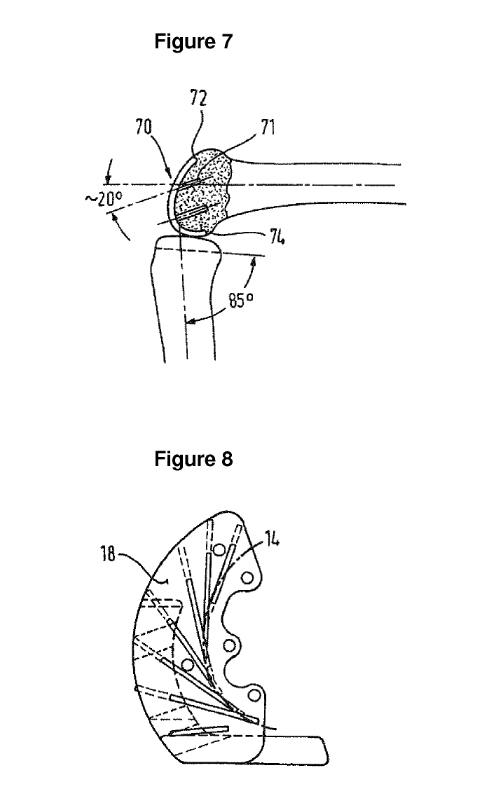

With guide holes 34, holes are preferably made in the outer contour to be generated with a drill so that pins 71 present on the joint endoprosthesis can be accommodated by the generated bone outer contour of the joint to be resected. As shown in FIG. 7, guide holes 34 are aligned in anterior guide body 30 so that with them holes can be generated in the outer contour to be generated which are preferably aligned approximately 20° to the longitudinal axis of the femur of the knee joint.

Guide slit 32 fulfils a similar function to guide holes 34 in that a bone-cutting tool introduced into it, such as, for example, a saw or a milling cutter, incorporates an additional groove into the outer contour of the bone to be generated. It is possible via a correspondingly formed groove to implant a joint endoprosthesis with a fin.

It is furthermore shown in FIGS. 2 to 6 that at least one contact point can also be embodied as a longitudinal projecting body. In the present embodiment there, a cylindrically embodied pin with contact point 42 is thus arranged substantially perpendicular to sagittal guide plate 10. Contact point 42 shown in FIGS. 2 to 4 and FIG. 6 furthermore has a marking 46 which is attached along its longitudinal direction.

Contact point 42 can likewise adjoin an anatomical point of orientation of the joint to be resected, but it is equally possible as shown in FIG. 5 that adjoining point 42 in one region adjoins the joint, where one of the ends of the joint endoprosthesis is located after implantation.

As shown in FIGS. 5 and 6, marking 46 serves to position cutting guide 1 at a fixed distance to joint 80. Cutting guide 1 converges with joint 80 until marking 46 and the outer contour of the joint to be resected lie on top of one another. As an alternative or in addition to the embodiments represented in the represented in the figures, the marking can also be provided at any desired other point of cutting guide 1 or can be formed by an edge, for example, an edge of anterior guide body 30.

1. A cutting guide for generating an outer contour on a bone, wherein a joint endoprosthesis, in particular a femoral component of a knee endoprosthesis, can be attached to the outer contour; the cutting guide has a sagittal guide plate which can be aligned with respect to the bone, and at least two sagittal guide slits for guiding a bone-cutting tool, the sagittal guide slits passing through the sagittal guide plate and being arranged at an angle to one another.

2. A cutting guide according to claim 1, wherein each sagittal guide slit forms a self-contained opening.

3. A cutting guide according to claim 1, wherein the guide slits on the sagittal guide plate are arranged substantially on an arcuate line and indeed preferably such that they are aligned with their longitudinal direction at an angle to the arcuate line and fan out towards the outside relative to the arcuate line.

4. A cutting guide according to claim 3, further comprising a transversal cleft plate which projects from the sagittal guide plate in the direction in which the guide slits of the sagittal guide plate extend through the sagittal guide plate.

5. A cutting guide according to claim 4, wherein there is located in the transversal cleft plate at least one further guide slit which extends through the transversal cleft plate so that it supplements the guide slits of the sagittal guide plate.

6. A cutting guide according to claim 5, further comprising an anterior guide body through which a guide slit which is substantially parallel to the sagittal guide plate extends and/or at least one guide hole.

7. A cutting guide according to claim 6, further comprising at least one alignment projection which is preferably arranged on the transversal cleft plate and which is suitable as a pivot point for alignment of the cutting guide in at least one spatial direction.

8. A cutting guide according to claim 7, further comprising the cutting guide has at least two contact points via which the cutting guide can be put in place, wherein at least one of the contact points projects.

9. A cutting guide according to claim 8, wherein the projecting contact point projects from the sagittal guide plate, the transversal cleft plate and/or the anterior guide body and a marking is present as a further positioning aid for the cutting guide at least one of the contact points.

10. A cutting guide according to claim 9, wherein it has at least one through-hole for fastening which is preferably arranged in the sagittal guide plate towards the inside relative to the arcuate line.

11. A cutting guide set for generating an outer contour on a knee joint, comprising two lateral and medial cutting guides according to claim 10 one of the preceding claims for at least one joint endoprosthesis size which are embodied in each case mirror-inverted.

12. The method for generating an outer contour on a bone for attachment of a joint endoprosthesis, in particular a femoral component of a knee endoprosthesis, by means of a cutting guide according to claim 11, the method comprising the following steps:
Putting in place and positioning the cutting guide on the lateral and/or medial side of the joint to be resected,
Alignment of the cutting guide relative to the joint,
Generating the outer contour with a bone-cutting tool,
Removing the cutting guide.

13. The method for generating an outer contour according to claim 12, wherein the cutting guide is put in place via the at least two contact points on the joint surface to be resected, wherein preferably at least one of the contact points is arranged on a transversal cleft plate which is pushed-in between the two sides of the joints.

14. The method for generating an outer contour according to claim 13, wherein the contact points of the cutting guide adjoin at least one of the following regions of the joint surface to be resected: the contact region where the opposing joint surfaces come into contact at approximately 85° flexion; the contact region where the opposing joint surfaces come into contact at approximately 0° extension; and/or the region in which one end of the joint endoprosthesis to be implanted, preferably the anterior end, rests against the bone.

15. The method for generating an outer contour according to claim 14, wherein, prior to putting in place of the cutting guide, a recess, preferably a step-shaped recess, is introduced into the opposite side of the joint.

16. The method for generating an outer contour according to claim 15, wherein the cutting device is aligned prior to generation of the outer contour via at least one alignment projection which serves as a pivot point.

17. The method for generating an outer contour according to claim 16, wherein the cutting guide, after putting in place, positioning and alignment via through-holes, is fastened on the bone of the joint to be resected.

18. The method for generating an outer contour according to claim 17, wherein a bone-cutting tool is introduced into the guide slits in order to resect the joint and at the same time, via the cuts carried out hereby, generate the desired outer contour for attachment of the joint prosthesis.

19. The method for generating an outer contour according to claim 18, wherein, prior to and/or after the generation of the outer contour for insertion of the prosthesis, via the at least one guide hole a hole and/or via the anterior guide slit a groove is introduced into the bone.
